(19) United States
(12) Patent Application Publication
(10) Pub. No.: US 2022/0039450 A1
(43) Pub. Date: Feb. 10, 2022

(54) SYNTHETIC NUTRITIONAL COMPOSITIONS TAILORED FOR INFANTS OF SPECIFIC AGES, AND NUTRITIONAL SYSTEMS COMPRISING THEM

(71) Applicant: SOCIETE DES PRODUiTS NESTLE S.A., Vevey (CH)

(72) Inventors: Carlos Antonio De Castro, Singapore (SG); Francesca Giuffrida, Mezieres (CH); Frederic Destaillats, Servian (CH); Sagar Thakkar, Tresalveo (SG)

(21) Appl. No.: 17/278,898

(22) PCT Filed: Sep. 24, 2019
(86) PCT No.: PCT/EP2019/075625

§ 371 Date: Mar. 23, 2021

(30) Foreign Application Priority Data
Sep. 25, 2018 (EP) 18196459.4

(51) Int. Cl. A23L 33/00; A23L 33/105

(52) U.S. Cl. CPC A23L 33/40; A23L 33/105

(57) ABSTRACT

Synthetic nutritional compositions tailored for infants of specific ages comprising Lutein in concentrations reflecting those found in human milk produced for infants of the same age (at the corresponding lactation stage), and nutritional systems comprising them.

### TECHNICAL FIELD

The invention relates to synthetic nutritional compositions tailored for infants of specific ages, to nutritional systems comprising them, and to their use to provide an optimised amount of lutein and/or one or more health benefit to an infant.

### BACKGROUND OF THE INVENTION

Even though breastfeeding is optimal for infants, the existence of certain conditions may mean that it is contraindicated. In such cases, where the sole source of nutrition is not available to infants, alternative strategies to feed them need to be employed. Feeding infants with synthetic nutritional compositions e.g. Infant formula is one such strategy.

The compositions of the aforementioned synthetic nutritional compositions e.g. infant formulas, aim to replicate those of human milk (hereinafter HM). However, replicating HM is not a simple task. HM not only contains numerous components, its composition is extremely dynamic and these dynamic changes remain largely unexplored and uncharacterized.

The inventors have now surprisingly found that the concentration of Lutein in HM may differ depending on the stage of lactation. Because such differences in the Lutein concentration of HM have never previously been identified, these differences are not reflected in the compositions of synthetic nutritional compositions available for infants today. Given that HM is considered the gold standard with respect to infant nutrition, there remains a need for synthetic nutritional compositions tailored for infants of specific ages which better reflect these identified differences.

### SUMMARY OF THE INVENTION

The invention is set out in the claims. The inventors have developed synthetic nutritional compositions tailored for infants of specific ages comprising Lutein in concentrations that reflect the concentrations of Lutein found in HM produced for infants of the same ages e.g. at corresponding lactation stages.

Said synthetic nutritional compositions may, for example, be an infant formula or a compositions for infants that are intended to be added to or diluted with human milk.

The Lutein concentration of said synthetic nutritional compositions tailored for infants of specific ages may differ. The Lutein concentration may be higher in a synthetic nutritional compositions tailored for infants of up to 4 months of age than in a synthetic nutritional compositions tailored for infants from 4 months of age. Further, the Lutein concentration may be higher in a synthetic nutritional compositions tailored for infants of up to 2 months of age than in a synthetic nutritional compositions tailored for infants from 2 month of age e.g. 2 month up to 4 months of age.

The synthetic nutritional compositions tailored for infants of specific ages may be included in a nutritional system.

The Lutein concentrations of the synthetic nutritional compositions of the invention more accurately reflect the Lutein concentrations found in HM produced for infants of the same ages i.e. at the corresponding stages of lactation. In light of this and, because HM is considered optimal with respect to infant nutrition, they, and the nutritional systems comprising them, can provide an optimized amount of Lutein to an infant, and may be used to ensure optimum Lutein levels in infants, for example an infant having the same age as the age of infant to which the synthetic nutritional composition is directed. The synthetic nutritional compositions may also be used to ensure optimum lutein intake and levels, and thereby to optimize antioxidant capacity as well as skin health and retinal development in said infants.

The synthetic nutritional compositions of the invention may be prepared from a non-age tailored synthetic nutritional composition by measuring out an appropriate amount of said non-age tailored synthetic nutritional composition and mixing it with an additive and/or diluent e.g. Lutein and/or water.

### DETAILED DESCRIPTION

The inventors performed a longitudinal study evaluating the nutrient composition of HM collected from mothers at various stages of lactation (30 days (1 month), 60 days (2 months), and 120 days (4 months) postpartum). Surprisingly, the inventors found that the results indicated that the concentration of Lutein found in HM can differ depending on the stage of lactation (when it is produced postpartum). In particular, the inventors found that the results indicated that the concentration of Lutein in HM may differ between HM produced up to 4 months postpartum and HM milk produced from 4 months postpartum. More particularly the inventors found that the results of the study indicated that the concentration of Lutein in HM produced up to 4 months postpartum may be higher than the concentration of Lutein in HM produced from 4 months postpartum. The study further indicated that the concentration of Lutein in HM produced up to 2 months postpartum may be higher than the concentration of Lutein in HM produced from 2 months postpartum e.g. 2 to 4 months postpartum.

Based on the findings of the study, the inventors have designed synthetic nutritional compositions tailored for infants of specific ages wherein, the Lutein concentration reflects that found in HM produced for an infant of the same age (at the corresponding lactation stage).

The term “synthetic nutritional composition tailored for an infant of a specific age” as used herein refers to any synthetic nutritional composition that is intended to be consumed by an infant of a specific age and, that is specifically adapted to the nutritional needs of an infant of said specific age.

Non limiting examples of synthetic nutritional compositions tailored for an infant from up to 4 months of age include; infant formulae, and a composition for infants that is intended to be added or diluted with HM e.g. HM fortifier.

Non limiting examples of synthetic nutritional compositions tailored for an infant of from 4 months of age include infant formulae, a composition for infants that is intended to be added or diluted with HM e.g. HM fortifier, or food stuffs intended for consumption by infants either alone or in combination with HM e.g. complementary foods.

The term “infant” as used herein refers to a human infant of 12 months of age or less.

In one embodiment, the infant according to the present invention is in need of lutein.

In another embodiment, the infant is in need of lutein and has non-optimal/sub-optimal lutein levels' intake.

In an aspect of the present invention there is provided a synthetic nutritional composition tailored for an infant of a specific age comprising lutein in a concentration reflecting that found in human milk produced for an infant of the same age (at the corresponding lactation age).

In one embodiment, the synthetic nutritional composition tailored for an infant of a specific age comprises, after reconstitution, a concentration of lutein reflecting that found in human milk produced for an infant of the same age (at the corresponding lactation age).

In an embodiment of the present invention said synthetic nutritional composition is tailored for an infant of a specific age selected from the group consisting of from 4 months of age and up to 4 months of age.

Non limiting examples of an age from 4 months of age include; 4, 5, 6, 7, 8, 9, 10, 11 and 12 months of age, 4 up to 6 months of age, 6 months to 9 months of age, 6 months to 12 months of age.

Non limiting examples of an age of up to 4 months of age include; up to 2 weeks of age, up to 1 month of age, 2 weeks to 1 month of age, up to 2 months of age, 1 month up to 2 months of age, from 2 months of age.

Non limiting examples of an age of up to 2 months of age include; up to 2 weeks of age, up to 1 month of age, 2 weeks to 1 month of age, up to 2 months of age, 1 month up to 2 months of age.

Non limiting examples of an age from 2 months of age include; 2 months to months of age, 2 months up to 3 months of age, 3 months up to 4 months of age, 3 months of age, 2 months of age; 4, 5, 6, 7, 8, 9, 10, 11 and 12 months of age, 4 up to 6 months of age, 6 months to 9 months of age, 6 months to 12 months of age.

Non limiting examples of an age from 2 months up to 4 months of age include; 2 months up to 3 months of age, 3 months up to 4 months of age, 3 months of age, 2 months of age.

In an embodiment the synthetic nutritional composition is tailored for an infant of up to 4 months of age and the Lutein concentration is 0.023 to 0.225 μg/ml, for example 0.024 to 0.167, 0.03 to 0.1, 0.05 to 0.075, 0.06 to 0.072 μg/ml.

The synthetic nutritional composition may, for example, be tailored for an infant of up to 2 months of age and may comprise Lutein in a concentration of 0.024 to 0.225 μg/ml for example, 0.06 to 0.072 μg/ml.

The synthetic nutritional composition may, for example, be tailored for an infant of from 2 months of age e.g. 2 months to 4 months of age and, may comprise Lutein in a concentration of 0.023 to 0.167 μg/ml, for example, 0.05 to 0.075 μg/ml.

In another embodiment the synthetic nutritional composition is tailored for an infant of from 4 months of age and the lutein concentration is 0.023 to 0.15 μg/ml, for example, 0.03 to 0.072, 0.04 to 0.06 μg/ml.

The Lutein concentration of the synthetic nutritional compositions tailored for an infant of a specific age as defined herein is expressed in μg/ml. This may refer to the Lutein concentration of a reconstituted synthetic nutritional compositions tailored for an infant of a specific age.

The term “lutein” as used herein refers to, free lutein, lutein esters, lutein salts and/or any combination of the foregoing. Free lutein refers to β,ε-carotene-3,3′-diol. Lutein esters and lutein salts respectively refer to esters or salts of β,ε-carotene-3,3′-diol. β,ε-carotene-3,3′-diol as referred to herein may be cis or trans or a mixture thereof.

The lutein concentration of a composition can be measured by methods well known in the art. For example, the lutein concentration of HM or a gender specific composition of the invention may be measured by extraction of the lutein with organic solvents e.g. BHT/Hexane/ethyl acetate. The analytical measurement of these extracted molecules may be done in two steps. The first step may be chromatographic separation by HPLC e.g. using isooctane/ethylacetate.

This step can be followed by second step of detection by diode array detectors and UV detectors. A method for the measurement of lutein in HM or in the gender specific composition as disclosed herein is set out in the examples included herein.

Any form of lutein suitable for administration to an infant to whom the synthetic nutritional composition is directed may be comprised within in the synthetic nutritional compositions of the invention. Lutein may for example be added as free lutein, lutein esters, lutein salts and/or any combination of the foregoing.

The lutein, in any form it is used, may stem from natural sources, in particular it may stem from animal or plant or algal sources of lutein that are either in free or esterified form.

The synthetic nutritional compositions tailored for an infant of a specific age can also comprise any other ingredients or excipients known to be employed in the type of synthetic nutritional composition in question e.g. infant formula, a composition for infants that is intended to be added to or diluted with human milk, HM fortifier, follow on formula, or food stuffs intended for consumption by infants e.g. complementary foods.

In an embodiment of the present invention the synthetic nutritional compositions tailored for an infant of a specific age are selected from the group consisting of: infant formula, and a composition for infants that is intended to be added to or diluted with human milk.

Non-limiting examples of ingredients known to be employed in the typed of synthetic nutritional compositions in question include: proteins, amino acids, carbohydrates, oligosaccharides, lipids, prebiotics or probiotics, essential fatty acids, nucleotides, nucleosides, other vitamins, minerals and other micronutrients.

Non limiting examples of proteins include: casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non-limiting examples of amino acids include leucine, threonine, tyrosine, Isoleucine, arginine, alanine, histidine, isoleucine, proline, valine, cysteine, glutamine, glutamic acid, glycine, serine, arginine, lysine, methionine, phenylalanine, tryptophane, asparagine, aspartic acid, and combinations thereof.

Non-limiting examples of carbohydrates include lactose, saccharose, maltodexirin, starch, and combinations thereof.

Non-limiting examples of lipids include: palm olein, high oleic sunflower oil, high oleic safflower oil, canola oil, fish oil, coconut oil, bovine milk fat, and combinations thereof.

Non-limiting examples of essential fatty acids include: linoleic acid (LA), α-linolenic acid (ALA) and polyunsaturated fatty acids (PUFAs). The nutritional compositions of the invention may further contain gangliosides monosialoganglioside-3 (GM3) and disialogangliosides 3 (GD3), phospholipids such as sphingomyelin, phospholipids phosphatidylcholine, phosphatidylethanolamine, phosphatidylinositol, phosphatidylserine, and combinations thereof.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof.

Non-limiting examples of Nucleotides include: cytidine monophosphate (CMP), uridine monophosphate (UMP), adenosine monophosphate (AMP), guanosine monophosphate (GMP), and combinations thereof.

Non limiting examples of other vitamins and minerals include: vitamin B1, vitamin B2, vitamin B6, vitamin B12, vitamin E. vitamin K. vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, L-carnitine, and combinations thereof. Minerals are usually added in salt form.

Other suitable and desirable ingredients of synthetic nutritional compositions, that may be employed in the synthetic nutritional compositions tailored for infants of specific ages, are described in guidelines issued by the Codex Alimentarius with respect to the type of synthetic nutritional composition in question e.g. Infant formula, HM fortifier, follow on formula, or food stuffs intended for consumption by infants e.g. complementary foods.

The synthetic nutritional compositions tailored for infants of specific ages may be prepared by methods well known in the art for preparing the type of synthetic nutritional composition in question e.g. infant formulae, follow on formulae, a composition for infants that is intended to be added or diluted with HM e.g. HM fortifier, or food stuffs intended for consumption by infants either alone or in combination with HM e.g. complementary foods.

An exemplary method for preparing a synthetic nutritional compositions tailored for infants of specific age that is age tailored powdered infant formula is as follows. A protein source, carbohydrate source, and fat source may be blended together in appropriate proportions. Emulsifiers maybe included in the blend. Vitamins and minerals (including lutein, for example as part of a vitamin premix) may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C.; for example by flash cooling. The liquid mixture may then be homogenised; for example in two stages at about 7 MPa to about 40 MPa in the first stage and about 2 MPa to about 14 MPa in the second stage.

The homogenised mixture may then be further cooled to add any heat sensitive components such as vitamins and minerals (if not added earlier, lutein may be added at this stage, for example as part of a vitamin premix). The pH and solids content of the homogenised mixture is conveniently standardised at this point.

The homogenised mixture can be transferred to a suitable drying apparatus such as a spray drier or freeze drier and converted to powder. The powder should have a moisture content of less than about 3% by weight.

If it is desired probiotic(s) may be added, they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the age tailored powdered infant formula by dry mixing.

The synthetic nutritional compositions tailored for an infant of a specific age may also be prepared from a non-age tailored synthetic nutritional composition.

In an aspect of the invention there is provided a method of preparing a synthetic nutritional compositions tailored for an infant of a specific age comprising: measuring out an appropriate amount of a non-age tailored synthetic nutritional composition and mixing it with an additive and/or a diluent e.g. water so as to arrive at a synthetic nutritional composition tailored to an infant of a specific age in accordance with the invention.

In one embodiment, the additive comprises lutein.

The additive may be an age tailored additive comprising Lutein in a particular concentration so that when mixed with the non-age specific synthetic nutritional composition, and optionally a diluent, the resulting mixture is a synthetic nutritional composition tailored to an infant of a specific age in accordance with the invention.

The non-age specific synthetic nutritional composition can be prepared by methods well known in the art for the type of composition in question e.g. as laid out above for infant formula.

One or more of the synthetic nutritional compositions tailored for an infant of a specific age can be included in a nutritional system.

The term “nutritional system” as used herein refers to a collection of more than one synthetic nutritional composition advertised or sold as part of the same product range e.g. a collection of infant formulas sold under the same brand and adapted to the nutritional needs of infants of differing ages and/or genders and/or delivered by different methods e.g. C-section. In one embodiment, the synthetic nutritional compositions making up the nutritional system are packaged individually e.g. in capsules or boxes. Said packages can be sold individually, grouped together e.g. wrapped by plastic film or combined in a box, or in a combination of these two ways. The nutritional system may also comprise synthetic nutritional compositions for children older than 12 months.

In a further aspect of the present invention there is provided a nutritional system comprising a synthetic nutritional composition tailored for an infant of a specific age in accordance with the invention.

In an embodiment the nutritional system comprises a synthetic nutritional composition tailored for an infant of up to 4 months of age, as disclosed herein, and a synthetic nutritional composition tailored for an infant from 4 months of age, as disclosed herein, wherein, the Lutein concentration of said synthetic nutritional composition tailored for an infant of up to 4 months of age is higher than that of said synthetic nutritional composition tailored for an infant from 4 months of age.

The Lutein concentration of the synthetic nutritional composition tailored for an infant of up to 4 months of age may be higher by any amount.

In an embodiment the synthetic nutritional composition tailored for an infant of up to 4 months of age comprises 0.0002 to 0.25 μg/ml, for example, 0.005 to 0.02, 0.01 to 0.018 μg/ml more Lutein than the synthetic nutritional compositions tailored for an infant from 4 months of age.

Non limiting examples of ages up to 2 months of age include; up to 2 weeks, up to 1 month, 1 month, 1 month up to 2 months, 2 months, 3 months, 2 to 4 months, 3 to 4 months of age.

Non limiting examples of an age from 4 months of age include; 4, 5, 6, 7, 8, 9, 10, 11, and 12 months of age, 4 to 6 months of age, 4 to 12 months of age, 6 to 12 months of age, 6 to 9 months of age, and 9 to 12 months of age.

In another embodiment the nutritional system comprises a synthetic nutritional composition tailored for an infant of up to 2 months of age and a synthetic nutritional composition tailored for an infant from 2 months of age e.g. 2 to 4 months of age, wherein, the Lutein concentration of said synthetic nutritional composition tailored for an infant of up to 2 months of age is higher than that of said synthetic nutritional composition tailored for an infant from 2 months of age.

The Lutein concentration of the synthetic nutritional composition tailored for an infant of up to 2 months of age may be higher by any amount.

In an embodiment the synthetic nutritional composition tailored for an infant up to 2 months of age comprises 0.001 to 0.2 μg/ml, for example, 0.007 to 0.06 μg/ml more Lutein than the synthetic nutritional compositions tailored for an infant from 2 months of age.

Synthetic nutritional compositions tailored for an infant of a specific age according to the invention are particularly suitable for use in a method of preparing single servings of infant formula using capsules, each capsule of which contains a unit dose of a synthetic nutritional composition e.g. an age tailored synthetic nutritional composition in a concentrated form, and which is equipped with opening means contained within the capsule to permit draining of the reconstituted synthetic nutritional composition directly from the capsule into a receiving vessel such as a baby bottle. Such a method is described in WO2006/077259.

The different synthetic nutritional compositions, including synthetic nutritional compositions tailored for an infant of a specific age may be packed into individual capsules and presented to the consumer in multipacks containing a sufficient number of capsules to meet the requirements of an infant of a particular age or age range, for one week for example. Suitable capsule constructions are disclosed in WO2003/059778.

The capsules can contain the synthetic nutritional compositions tailored for an infant of a specific age in the form of powders or concentrated liquids in both cases for reconstitution by an appropriate amount of water. The synthetic nutritional compositions tailored for an infant of a specific ages and/or the quantity of a synthetic nutritional compositions e.g. infant formula in the capsules may vary according to age of the infant. If necessary, different sizes of capsules may be provided for the preparation of infant formulas for infants of different ages.

Because HM is the gold standard when it comes to infant nutrition, and because the Lutein concentration of the synthetic nutritional compositions of the invention better reflect the Lutein concentrations found in HM at corresponding lactation stages, they, and the nutritional systems comprising them, may be used to provide an optimum amount of Lutein to an infant and to ensure optimum Lutein levels.

Lutein is a lipophilic nutrient that is necessary for retina development. It also has also role in skin health and, has antioxidant capacity.

In another aspect of the present invention there is provided a specific synthetic nutritional composition of the invention and/or a nutritional system of the invention for use to prevent and/or treat non-optimal/sub-optimal lutein levels in an infant for example an infant up to 4 months of age, or an infant from 4 months of age.

In another aspect of the present invention there is provided the use of a specific synthetic nutritional composition of the invention to provide an optimum amount of lutein to an infant, to optimize antioxidant capacity, to optimize skin health and/or, to optimize retinal development, for example, in an infant up to 4 months of age, or from 4 months of age.

In another aspect of the present invention there is provided the use of a nutritional system of the invention to provide an optimum amount of lutein to an infant, to optimize antioxidant capacity, to optimize skin health and/or, to optimize retinal development, for example, in an infant up to 4 months of age, or from 4 months of age.

The nutritional system may provide an optimum amount of Lutein to an infant up to 12, 11, 10, 9, 8, 7, 6, 5, 4, 3, 2, 1 months of age and/or up to 2 weeks of age.

In another aspect of the present invention there is provided a method treating and/or preventing sub-optimal lutein levels in an infant or for providing an optimum amount of lutein to an infant comprising:
a) Optionally preparing an synthetic nutritional composition, tailored for an infant of a specific age from an non-age tailored synthetic nutritional composition;
b) Feeding a synthetic nutritional composition tailored for an infant of a specific age to an infant, in particular to an infant of an age for which the said synthetic nutritional composition is tailored for example an infant of up to 4 months of age, or an infant from 4 months of age.

As stated herein, a synthetic nutritional composition tailored for an infant of a specific age, according to the invention, may be prepared from a non-age tailored synthetic nutritional composition. Accordingly, in another aspect of the present invention there is provided a kit for providing an optimized amount of Lutein to an infant comprising:
a) An non-age tailored synthetic nutritional composition
b) A label indicating dosage requirements for said non-age tailored synthetic nutritional composition so as to arrive at a synthetic nutritional composition tailored for an infant of a specific age in accordance with the invention.

The dosage requirements may be with respect to the quantity of the non-age tailored synthetic nutritional composition employed and/or the consumption frequency e.g. 4 times per day.

The kit and method may provide an optimized amount of Lutein to an infant, in particular an infant up to 4 months, or an infant from 4 months age.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Longitudinal Clinical Trial:

The present inventors designed a longitudinal clinical trial with 50 lactating mothers with milk sampling at 30 (visit 1), 60 (visit 2) and 120 (visit 3) days post-partum. The milk samples were quantitatively analyzed for lutein.

Human milk collection: The protocol and collection of human milk was reviewed and approved by the local ethical committee of Singapore. The study took place at National University of Singapore. Volunteer mothers of term infants, who were apparently healthy and non-smokers (n=50; 31.1±3.1-year old) provided breast milk samples (approximately 30 mL). Samples were collected after full expression from one breast using a milk pump, while the baby was fed on the other breast. All efforts were made to collect complete feed that included fore-milk, mid-milk and hind-milk as a representation of one feed, to avoid within feed variation of lipid content. Approximately 30 mL aliquot was separated in a conical polypropylene tube for this study and the rest was fed to the infant. Samples collected for research were stored at −80° C. until analyses. Data collection points were 30 days (1 month), 60 days (2 months) and 120 days (4 months) postpartum.

Measurement of the Lutein Concentrations in Samples:

The lutein concentration of each sample was measured by extraction of the lipids and lipophilic molecules by organic solvents. The analytical measurement of these extracted molecules was done in two steps. The first step was chromatographic separation by HPLC followed by second step of detection by diode array detectors and UV detectors.

Material and Methods

Chemicals

Lutein (≥75%) was purchased from Merck/Sigma-Aldrich (Darmstadt, Germany). ULC/MS grade water and absolute methanol produced by Biosolve were purchased from Chemie Brunschwig AG (Basel, Switzerland). HPLC-grade water was prepared using a Millipore Milli-Q purification system (EMD Millipore, Billerica, Mass.). All other HPLC-grade solvents and reagents were purchased from Merck/Sigma-Aldrich (Darmstadt, Germany).

Preparation of Standard Solutions

All preparations took place in a laboratory where the light source was equipped with UV filters to avoid degradation of light-sensitive carotenoids. To ensure accuracy, positive displacement pipettes (Microman, Gilson®) were used in the preparation of all standard and calibration solutions.

Standard solutions. Stock standard solutions were individually prepared at target concentrations by dissolving lutein in the appropriate solvent (see Table A).

TABLE A

| | | Dilution | | | | |
|---|---|---|---|---|---|---|
| | | solvent | Dilution | | | |
| | Stock | for stock | solvent for | Dilution | Wavelength | |
| Compound | solution | solution | measurement | factor | (nm) | E_{cm}^{1%} |
| Lutein | 1 mg/10 mL | Methylene | Ethanol | 40 | 445 | 2550 |
| | | chloride | | | | |

All the solutions were stored in 2 mL aliquots at −18° C. until use for a maximum of 3 months or 1 year in the case of labelled internal standards. The concentration of each of the standard stock solution was determined by spectrophotometry on a spectrophotometer. For this purpose, an aliquot of each solution was individually pipetted into an amber-glass volumetric flask, evaporated to dryness under a nitrogen stream and dissolved in the appropriate solvent (Table A). UV absorbance of the resulting solutions were measured against reference solvent and extinction coefficients used to back calculate the concentration of stock solutions. Chromatographic purity was also determined. 200 μL of the solutions used for spectrophotometric purity determination were dried down in a HLPC vial and dissolved in 100 μL of isooctane-ethyl acetate (90:10) for injection into the LC system.

Standard stock solutions were combined into a standard intermediate solution pipetting each of the individual stock solutions into a 20-mL amber glass volumetric flask, drying down under a nitrogen stream at room temperature and dissolving in isooctane-ethyl acetate. Final concentrations were 0.5 μg/mL for lutein.

Standard solutions for calibration. Calibrating solutions were prepared by pipetting into individual 2-mL HPLC amber vials a series of different volumes of working standard solutions and a fixed volume (20 μL) of internal standard working solution to provide an extended calibration range. After evaporation to dryness, the residue was dissolved into 100 μL of isooctane-ethyl acetate (90:10) for the analysis of lutein.

Sample Preparation

Into an 8-mL glass tube, 5 μL of an ethanolic solution of butylated hydroxytoluene (BHT) (79 g/L), 10 μL of an aqueous solution of deferoxamine mesylate (10 mg/mL), 1 mL ethanol, and 25 μL internal standard working solution were added successively to 750 μL of human milk and mixed. Then, 2.5 mL of n-hexane:ethyl acetate (90:10) (v/v) containing 350 mg/L BHT was added and mixed vigorously in a multitube shaker for 2 minutes in pulse mode and 2 minutes in continuous mode. The tubes were centrifuged at 2500 rpm/min for 10 min at 4° C. and the upper organic phase collected into a clean glass tube. The liquid/liquid extraction process was repeated, the supernatants were combined in the same 8-mL tube and taken down to almost dryness under a nitrogen stream. The residue was quantitatively transferred into a 2-mL microcentrifuge tube using small portions of n-hexane/ethyl acetate 90:10 (v/v), dried and dissolved in 125 μL of isooctane:ethyl acetate 90:10 (v/v). The extracts were centrifuged at 11′000 rpm/min for 10 minutes at room temperature and transferred into low volume HPLC vials for analysis of lutein.

Chromatographic Analysis

Lutein was analyzed in Normal Phase LC mode using a Hypersil GOLD™ Silica, 1.9 μm, 200×2.1 mm column equipped with a 0.2 μm in-line filter (Thermo, Switzerland). The chromatography system consisted in a Waters Acquity UPLC® system equipped with a photodiode array (PDA) eLambda and a Fluorescence Detector (Waters, Baden, Switzerland). The chromatographic column was kept at 35° C. through analysis. Solvent A was n-hexane for chromatography and solvent B a mix of n-hexane-dioxane 50:50 (V/V) containing 0.01% acetic acid. A gradient of solvent B was applied, starting from 28% at time=0 min ramping to 40% in 2 minutes, to 40% at minute 5 with a constant flow rate of 0.4 mL/min. This was followed by a ramp of 1 minute to 100% solvent B and reduced flow rate of 0.3 ml/min for a 2 minutes cleaning step. The analytical column was slowly requilibrated by increasing both solvent A and flow rate to initial conditions in 2 minutes for a total run time of 10 minutesl. Data were collected and processed using Waters Empower™ software. Calibration and quantification were performed using a linear regression and external standards were analyzed. A gradient of solvent B was applied, starting from 0.5% at time=0 min ramping to 2% in 2 minutes, to 10% at minute 5, 45% at minute 10 and 50% at 12 minutes with a constant flow rate of 0.4 mL/min. This timeframe was followed by a ramp of 1 minute to 100% solvent B and reduced flow rate of 0.3 ml/min for a 2 minutes cleaning step. The analytical column was slowly requilibrated by increasing both solvent A and flow rate to initial conditions in 2 minutes and stabilized 5 minutes for a total run time of 22 minutes. Injection volume was 5 μL. PDA detector recorded signals at 450 nm for lutein. Data were collected and processed using Waters Empower™ software. Calibration and quantification were performed using linear regression with apocarotenal.

The results of the analysis of the HM, with respect to Lutein concentration, are shown table I.

TABLE I

| | | | Lutein Concentration μg/mL | | | |
|---|---|---|---|---|---|---|
| Stage | | Min | Mean | Median | Max | SD |
| 30 | days | 0.02475662 | 0.071153 | 0.06027 | 0.2248269 | 0.047988 |
| 60 | days | 0.02324095 | 0.074877 | 0.052981 | 0.16704935 | 0.048922 |
| 120 | days | 0.02307577 | 0.057642 | 0.047568 | 0.14293318 | 0.031795 |

### Example 2

Examples of synthetic nutritional compositions (infant formulas) tailored to infants of specific ages are given in table III

TABLE III

| | up to | 2 months up | from |
|---|---|---|---|
| | 2 months | to 4 months | 4 months |
| | of age | of age | of age |
| Ingredients | Per Litre | Per Litre | Per Litre |
| Energy (kcal) | 670 | 670 | 670 |
| Protein (g) | 12.1 | 12.1 | 12.1 |
| Fat (g) | 35.639 | 35.639 | 35.649 |
| Cholesterol (g) | 0.061 | 0.061 | 0.051 |
| Linoleic acid (g) | 5.3 | 5.3 | 5.3 |
| α-Linolenic acid (mg) | 675 | 675 | 675 |
| Lactose (g) | 74.7 | 74.7 | 74.7 |
| Prebiotic (100% GOS) (g) | 4.3 | 4.3 | 4.3 |
| Minerals (g) | 2.5 | 2.5 | 2.5 |
| Na (mg) | 150 | 150 | 150 |
| K (mg) | 590 | 590 | 590 |
| Cl (mg) | 430 | 430 | 430 |
| Ca (mg) | 410 | 410 | 410 |
| P (mg) | 210 | 210 | 210 |
| Mg (mg) | 50 | 50 | 50 |
| Mn (μg) | 50 | 50 | 50 |
| Se (μg) | 13 | 13 | 13 |
| Lutein (μg) | 61 | 53 | 48 |
| Vitamin D (μg) | 10 | 10 | 10 |
| Vitamin E (mg TE) | 5.4 | 5.4 | 5.4 |
| Vitamin K1 (μg) | 54 | 54 | 54 |
| Vitamin C (mg) | 67 | 67 | 67 |
| Vitamin B1 (mg) | 0.47 | 0.47 | 0.47 |
| Vitamin B2 (mg) | 1 | 1 | 1 |
| Niacin (mg) | 6.7 | 6.7 | 6.7 |
| Vitamin B6 (mg) | 0.5 | 0.5 | 0.5 |
| Lactoferrin (bovine) g | 1 | 1 | 1 |
| Folic acid (μg) | 60 | 60 | 60 |
| Pantothenic acid (mg) | 3 | 3 | 3 |
| Vitamin B12 (μg) | 2 | 2 | 2 |
| Biotin (μg) | 15 | 15 | 15 |
| Choline (mg) | 67 | 67 | 67 |
| Fe (mg) | 8 | 8 | 8 |
| I (μg) | 100 | 100 | 100 |
| Cu (mg) | 0.4 | 0.4 | 0.4 |
| Zn (mg) | 5 | 5 | 5 |

### Example 3

An example of a nutritional system in accordance with the invention is given in table IV.

TABLE IV

| | Up to | from |
|---|---|---|
| | 4 months | 4 months |
| | of age | of age |
| Ingredients | Per Litre | Per Litre |
| Energy (kcal) | 670 | 670 |
| Protein (g) | 12.1 | 12.1 |
| Fat (g) | 35.639 | 35.649 |
| Cholesterol (g) | 0.061 | 0.051 |
| Linoleic acid (g) | 5.3 | 5.3 |
| α-Linolenic acid (mg) | 675 | 675 |
| Lactose (g) | 74.7 | 74.7 |
| Prebiotic (100% GOS) (g) | 4.3 | 4.3 |
| Minerals (g) | 2.5 | 2.5 |
| Na (mg) | 150 | 150 |
| K (mg) | 590 | 590 |
| Cl (mg) | 430 | 430 |
| Ca (mg) | 410 | 410 |
| P (mg) | 210 | 210 |
| Mg (mg) | 50 | 50 |
| Mn (μg) | 50 | 50 |
| Se (μg) | 13 | 13 |
| Lutein (μg) | 60 | 48 |
| Vitamin D (μg) | 10 | 10 |
| Vitamin E (mg TE) | 5.4 | 5.4 |
| Vitamin K1 (μg) | 54 | 54 |
| Vitamin C (mg) | 67 | 67 |
| Vitamin B1 (mg) | 0.47 | 0.47 |
| Vitamin B2 (mg) | 1 | 1 |
| Niacin (mg) | 6.7 | 6.7 |
| Vitamin B6 (mg) | 0.5 | 0.5 |
| Lactoferrin (bovine) g | 1 | 1 |
| Folic acid (μg) | 60 | 60 |
| Pantothenic acid (mg) | 3 | 3 |
| Vitamin B12 (μg) | 2 | 2 |
| Biotin (μg) | 15 | 15 |
| Choline (mg) | 67 | 67 |
| Fe (mg) | 8 | 8 |
| I (μg) | 100 | 100 |
| Cu (mg) | 0.4 | 0.4 |
| Zn (mg) | 5 | 5 |

1. A synthetic nutritional composition tailored for an infant of a specific age comprising Lutein in a concentration reflecting that found in human milk produced for an infant of the same age.

2. A synthetic nutritional composition according to claim 1 wherein the synthetic nutritional composition is tailored for an infant of a specific age selected from the group consisting of up to 4 months of age, and from 4 months of age.

3. A synthetic nutritional composition according to claim 2 wherein when the synthetic nutritional composition is tailored for an infant up to 4 months of age the Lutein concentration is 0.023 to 0.225 mg/ml, and when the synthetic nutritional composition is tailored for an infant of from 4 months of age the lutein concentration is 0.023 to 0.15 mg/ml.

4. A synthetic nutritional composition tailored for an infant of a specific age according to claim 1 selected from the group consisting of: infant formula, and a composition for infants that is intended to be added to or diluted with human milk.

5-11. (canceled)

12. A method for providing an optimum amount of Lutein to an infant comprising:
a. preparing a synthetic nutritional composition tailored for an infant of a specific age from an non-age specific synthetic nutritional composition comprising Lutein;
b. Feeding the synthetic nutritional composition tailored for an infant of a specific age to an infant of an age for which the synthetic nutritional composition is tailored.

13. (canceled)

14. A kit for providing an optimized amount of Lutein to an infant, the kit comprising:
a. An non-age specific synthetic nutritional composition
b. A label indicating dosage requirements for an infant so as to arrive at a synthetic nutritional composition tailored for an infant of a specific age comprising Lutein in a concentration reflecting that found in human milk produced for an infant of the same age.

15-16. (canceled)
